(19) United States
(12) Patent Application Publication
(10) Pub. No.: US 2013/0035303 A1
(43) Pub. Date: Feb. 7, 2013

(54) COMPOSITIONS AND METHODS FOR CONTROL OF SAND FLIES AND OTHER BLOOD SUCKING INSECTS

(71) Applicant: Richard Poche, Wellington, CO (US)

(72) Inventor: Richard Poche, Wellington, CO (US)

(73) Assignee: Genesis Laboratories, Inc., Wellington, DE (US)

(21) Appl. No.: 13/641,848

(22) PCT Filed: Apr. 21, 2011
(86) PCT No.: PCT/US11/33415

§ 371 Date: Oct. 17, 2012

Related U.S. Application Data
(60) Provisional application No. 61/326,920, filed on Apr. 22, 2010.

(51) Int. Cl. A61K 31/7048; A61P 33/14; A61P 33/00

(52) U.S. Cl. 514/30

(57) ABSTRACT

The invention relates to a new rapid release oral formulation of fipronil or imidacloprid for the effective control of blood-sucking insect populations. Embodiments of the invention relate to their use by incorporation into a feed-through formulation that can be administered orally to host animals such as birds, goats, dogs, and cattle for the rapid effective control of blood sucking insects. The formulation is fast acting and the residue of the chemicals present in the feces serves as a larvacide, effectively controlling newly hatched larvae.

### RELATED CASES

Claims priority to U.S. application Ser. No. 61/326,920, filed Apr. 22, 2010, and entitled “A novel method for the control of sand flies and other blood sucking insect populations”, incorporated by reference herein in its entirety.

### FIELD

This disclosure relates to compositions and the use of such compositions to control blood sucking insects. In particular, insecticidal chemicals incorporated into feed-through formulations can be administered orally to host animals for the rapid uptake and effective population control of blood sucking insects.

### BACKGROUND

The statements in this section merely provide background information related to the present disclosure and may not constitute prior art.

In the tropical world, mosquitoes (Anopheles spp.), tsetse flies (Glossina spp.), and sand flies (Phlebotomus spp. and Lutsomia spp.), among others, serve as important vectors in transmitting devastating diseases, such as Malaria, dengue, yellow fever, chikangunya, and cutaneous and visceral leishmaniasis. These diseases are responsible for most of the preventable deaths in poor regions of the world. The insects that serve as vectors for these diseases are typically classified as sucking and biting insects that require a blood meal from a warm blooded mammal during egg laying.

Leishmaniasis is a vector-facilitated parasitic infection affecting 350 million people worldwide. Twenty species of Leishmania are transmitted by approximately 30 proven phlebotomine vectors to 1.5-2 million people in 88 countries annually. In the old world, the Leishmania parasite is transmitted by members of the genus Phlebotomus from either anthroponotic or zoonotic reservoirs (Desjeux, 1996; Desjeux, 2004; Alvar, 2006).

Visceral leishmaniasis (VL), generally known as kala-azar on the Indian subcontinent, is caused by Leishmania donovani and is the most severe clinical form of the leishmaniasis. Approximately 500,000 of leishmaniasis cases contracted annually are VL, over 90% of which occur in impoverished areas of Bangladesh, Brazil, India, Nepal, and Sudan, and half of which are located on the Indian subcontinent, primarily within Bihar state (Desjeux, 2001; Bern et al., 2005; Singh et al., 2006; Dey et al., 2007). VL is largely considered a rural disease, often correlated with malnutrition, poor sanitary conditions, and other factors associated with low socioeconomic status. Studies indicate an increased risk for urbanized areas as livestock populations increase and no prophylactics or vaccinations are available at present (Desjeux, 2001, 2002, 2004; Coleman et al., 2006).

Control of disease vector insects has been the subject of patents and publications resulting in dozens of effective insecticidal chemicals targeted to the control of blood-sucking insects. Numerous formulations have been devised to selectively deliver these insecticidal compounds in the field for the most effective insect population control. Specifically, one of the most effective, area-wide control of these insects is achieved by killing the adult insects while they are feeding on a host animal, ideally while simultaneously controlling the hatching of insect larvae, which typically feed in the animal's feces. Previously described insecticides that have been used as pour-ons, injectables, or oral products for treatment of livestock are the avermectins such as ivermectin and eprinomectin.

Historic measures of VL vector control in India, Bangladesh, and Nepal are limited primarily to broadcast application of DDT. A byproduct of systematic spray programs focused on malaria control initiated in the 1950s included a sharp decline in sand fly populations (Choudhury and Saxena, 1987; Killick-Kendrick, 1999). Despite a lack of sustainability due to logistical difficulties and the excessive cost of program maintenance, indoor residual spraying (IRS) continues to be the primary form of Leishmania vector control in India (Desjeux, 2004). Additionally, data is becoming increasingly prevalent about the tolerance of phlebotomine species to commonly utilized insecticides such as DDT, malathion, and permethrin (Dinesh et al., 2001; Tetreault et al., 2001; Barnet et al., 2005; Kumar et al., 2009). Alternative methods of suppressing VL transmission rates include treated bed net campaigns and plastering of mud floors and walls of homes and cattle sheds. However the limited successes with these methods are seemingly incidental and most studies show clear indications that application of these alternative methods is impractical (Kishore et al., 2006; Joshi et al., 2009).

A number of publications have described the use of these insecticidal compounds as applied to cattle, goats and other live-stock, for the control of blood-sucking insects.

Williams et al. in WO 99/027906 mention that fipronil, avermectins, and other insecticides and parasiticides have been formulated into long-acting injectable formulations for the treatment of parasitic infestations in cattle and other live-stock.

Yao et al. in CN 20091069402 mention a slow-release avermectin tablet for use in livestock and poultry for the control of flies and fleas.

Yuwan et al. in CN 19981024497 mention the use of an anti-parasite oral spray containing ivermectin for sheep.

Rowe et al. in US 20050047923 mention the use of an anti-parasite oral spray containing ivermectin for sheep. Although developed as an anthelmintic it also showed some low ectoparasitic efficacy.

Poche et al. in US 2006057178 mention the “simultaneous” control of rodents and at least one insect pest (e.g., cockroach, ants, ticks) through the same bait incorporating insecticides such as imidacloprid or fipronil and a rodenticide.

Other oral formulations used to treat mammals for worms and other parasites are described by Freehauf et al. in NZ 537407.

Still more oral formulations used to treat mammals for worms and other parasites are mentioned in WO 2007/075827, wherein a homogenous oral veterinary paste is used to deliver the active insecticidal agents.

Furstenau et al. in NZ 314603 mention a triglyceride oil based oral drench containing avermectin and stabilizing agents.

Wood et al. in US 19890316625 mention another type of oral delivery system using a bolus with a sustained release formulation for the oral administration of parricidal agents.

Most recently, Johnson et al. in WO 2010/039892 mention the systemic treatment of blood-sucking and blood-consuming parasites by the oral administration of insecticides.

However, in all of the above representative disclosures, the rapid and sustained efficacy of ectoparasitic control of blood-sucking insects using an oral delivery system has not been addressed. Livestock in tropical regions typically graze unsupervised, often close to human dwellings, and are housed near human dwellings. As such, control of blood-sucking insects to prevent the spread of diseases is also required in the immediate vicinity of human dwellings. Thus an animal fed a bolus or other oral delivery formulation of an ectoparasitic compound should ideally begin to exhibit insecticidal effect within the first twenty four hours of treatment to ensure the maximum control of insect populations close to human dwellings.

The present invention is directed toward overcoming one or more of the problems discussed above.

### BRIEF DESCRIPTION OF THE DRAWINGS

The drawings described herein are for illustration purposes only and are not intended to limit the scope of the present disclosure in any way.

FIG. 1 shows percent mortality of one to two day old first instar P. argentipes larvae fed feces from five treatment groups of insecticide treated R. rattus, R. rattus control, and standard larval food control. Fecal samples are from day 1 post-treatment.

FIG. 2 shows mortality of P. papatasi larvae due to imidacloprid.

FIG. 3 shows percent mortality of one to two day old first instar P. argentipes larvae fed feces from three treatment groups of fipronil treated B. bengalensis, B. bengalensis control, and standard larval food control. Fecal samples are from day 1 post-treatment.

FIG. 4 shows percent mortality of one to two day old first instar P. argentipes larvae fed feces from three treatment groups of fipronil treated B. bengalensis, B. bengalensis control, and standard larval food control. Fecal samples are from day 5 post-treatment.

FIG. 5 shows percent mortality of one to two day old first instar P. argentipes larvae fed feces from three treatment groups of fipronil treated B. bengalensis, B. bengalensis control, and standard larval food control. Fecal samples are from day 10 post-treatment.

FIG. 6 shows percent mortality of one to two day old first instar P. argentipes larvae fed feces from three treatment groups of fipronil treated B. bengalensis, B. bengalensis control, and standard larval food control. Fecal samples are from day 20 post-treatment.

FIG. 7 shows percent mortality of adult P. argentipes following bloodfeeding on treated B. bengalensis 20 days following rodent treatment.

FIG. 8 shows day 14 post-treatment adult sand fly mortality results after one hour exposure to cattle receiving different dose levels of fipronil.

FIG. 9 shows day 21 post-treatment adult sand fly mortality results after one hour exposure to cattle receiving different dose levels of fipronil.

FIG. 10 shows P. argentipes larval mortality when exposed to D-14 fipronil treated cattle feces.

FIG. 11 shows P. argentipes larval mortality when exposed to D-21 fipronil treated cattle feces.

### SUMMARY

Provided herein are compositions and methods for the use of such compositions to control blood sucking insects. In some embodiments, insecticidal chemicals incorporated into feed-through formulations are administered orally to host animals for the rapid uptake and effective population control of sand flies, mosquitoes, tsetse flies, and other blood sucking insects including arachnids such as ticks. The compositions are fast acting and the residue of the chemicals present in the feces serves as a larvacide, effectively controlling newly hatched larvae.

Surprisingly, compositions provided herein comprising the ectoparasitic compounds fipronil or imidacloprid, when formulated into a quick uptake oral delivery formulation, demonstrate unexpectedly quick control of blood-sucking insects. Further, this quick action results in improved reduction in a localized population of blood-sucking insects, for example, sand flies that carry the Leishmania causing parasite. Still further, the oral delivery compositions are surprisingly effective in increasing concentration of the pesticide in the feces where the insects lay their eggs.

In many countries, cattle are washed daily in hot weather, thus removing any dermally applied insecticides. Oral formulations, such as the bolus or tablet, are more practical to ensure quick absorption of the drug.

Topical fipronil is slowly absorbed by the tissues from the circulatory system and because the active is lipophilic, the compound is sequestered by the body's fat stores and released back into circulation over time.

This quick uptake formulation incorporating the chemicals fipronil, imidacloprid, or ivermectin (or abamectin, doramectin, eprinomectin, or amamectin) in combination with fipronil when added into a feed-through product or bolus is effective in controlling sand fly species, as well as mosquitoes and tsetse flies. Within several hours, when administered orally to a host animal, these chemicals are absorbed into the blood and are efficacious against adult biting flies. In addition, residues of these chemicals that end up in the feces of treated animals serve as larvacides and control newly hatched larvae. Data from both the laboratory and field demonstrate excellent efficacy on sand flies, both adults and larvae, as well as other biting insects. Compositions comprising the fipronil in combination with ivermectin (and/or other like compounds) are useful in controlling both ectoparasites and endoparasites.

Further areas of applicability of the present teachings will become apparent from the description provided herein. It should be understood that the description and specific examples are intended for purposes of illustration only and are not intended to limit the scope of the present teachings.

### DETAILED DESCRIPTION

The following description is merely exemplary in nature and is not intended to limit the present disclosure, application, claims, compositions, or uses.

Insect Growth Regulators (IGR) such as diflubenzuron, cyromazine, and novaluron and insecticides such as fipronil, imidacloprid, and cyfluthrin are typical pour-on pesticides, formulated as granular or wettable powders, and are not orally administered.

Disclosed herein are compositions and methods of using those compositions to control bloodsucking insects. Surprisingly, fipronil or imidacloprid, when formulated into quick uptake oral delivery compositions, dramatically controls the localized population of bloodsucking insects. The inventors conceived of insecticidal formulations that concurrently control adult insect infestations and larval infestations in feces.

The compositions and methods described herein are useful for a variety of animals, including mammals and birds, for example, avian, simian, bovine, canine, equine, asinine, feline, hicrine, murine, ovine, cameline, camelid, leporine, macropodine, human, galline, and porcine animals. In some embodiments, the animal is a canine or feline. In other embodiments, the animal is a bovine. In still other embodiments, the animal is an ovine, an hircine, or a caprine. In still further embodiments, the animal is a galline.

Ectoparasites are parasites that typically live on the surface of the host. As used herein, the term “ectoparasite” is used interchangeably with the phrase “bloodsucking parasite” or with the phrase “bloodsucking insect”. Exemplary ectoparasites include fleas, lice, ticks, sand flies, deer flies, horse flies, stable flies, mosquitoes, bedbugs, blowflies, louseflies, blackflies, tsetse flies, bloodsucking conenose, and mites. Many diseases are carried by microorganisms dependent on ectoparasites for part of their lifecycle.

Likewise, mosquitoes transmit malaria (plasmodium parasites); flaviviruses which cause yellow fever, dengue fever, Japanese encephalitis, West Nile infection, and St. Louis encephalitis; alphaviruses which cause equine encephalitis and chikangunya; bunyaviruses which cause LaCrosse encephalitis, reoviruses, Rift valley fever, and Colorado tick fever.

Control of bloodsucking parasites using the compositions and methods described herein will improve the quality of life of the animals typically infected by the parasites, improve the productivity of these animals (improved weight gain, increased live births, increased birth weights, improved milk production, etc.), and improve the quality of life of the humans who come into contact with the animals.

Thus, the inventors have determined that oral administration of certain insecticides allows for quick uptake of the insecticide and surprisingly good control of the target insect population. For example, oral administration of an imidacloprid or fipronil composition to livestock surprisingly controls sand flies, both by control of the adult population which ingests the insecticide by sucking the blood of a treated animal, and by control of the larval population which cannot survive in manure from a treated animal (as the manure contains larvicidal levels of insecticide). As mentioned above, sand flies (Phlebotomus spp. and Lutsomia spp.) serve as important vectors in transmitting the devastating disease of cutaneous and visceral leishmaniasis which is responsible for many preventable deaths in poor regions of the world. Control of the host insect population will effectively control spread of leishmaniasis, both in humans and animals.

Compositions typically used in the treatment or control of bloodsucking parasites on animals do not act quickly enough to provide effective insect population control. It is desirable that the insecticidal composition exhibits insecticidal effect within several hours of treatment. Compositions conceived by the inventors herein exhibit insecticidal effect within hours of treatment, for example, within about 12 hours of treatment, within about 10 hours of treatment, within about 8 hours of treatment, within about 6 hours of treatment, within about 4 hours of treatment, within about 2 hours of treatment, within about 1 hour of treatment, or within about 30 minutes of treatment. As used herein, the phrase “insecticidal effect” is used to indicate insect mortality due to treatment of a host animal with an insecticide. In some aspects, the “insecticidal effect” is absolute. In other aspects, the “insecticidal effect” is relative.

In one aspect, the composition is an oral formulation comprising one or more insecticides such as fipronil or imidacloprid. In some embodiments, the composition is an oral formulation comprising an insecticide and one or more ingredients suitable for consumption by a mammal. Surprisingly, such compositions when fed to a mammal exhibit quick insecticidal effect relative to pour-on and spray-on (or drench) insecticide formulations.

The compositions are administered orally using any suitable form for oral administration, e.g., tablets, pills, suspensions, solutions (possibly admixed with drinking water), emulsions, capsules, powders, syrups, and palatable feed compositions. In some embodiments, the insecticide and other ingredients are admixed during manufacture process used to prepare the composition. The compositions can be fed directly to the animal as a treat or can be added to feed compositions during or after the manufacturing of the feed composition.

The insecticide can be incorporated into the composition during the processing of the formulation, such as during and/or after mixing of other components of the composition. Distribution of these components into the composition is accomplished by conventional means. Unless otherwise specifically indicated, all weights and concentrations for the compositions of the present invention are based on dry weight of a composition after all components and ingredients are admixed.

In some embodiments, the composition is a food. Both liquid and solid foods are contemplated herein. When the food is a liquid, the insecticide may be admixed with the food or with water. Where the food is solid, the insecticide may be coated on the food, incorporated into the food, or both. The food includes both dry foods and wet foods. The non-insecticidal components of the food and their typical proportions are known to skilled artisans and typically include carbohydrates, proteins, fats, fibers, and/or nutritional ingredients such as vitamins, minerals, and the like.

Illustratively, the insecticidal composition can be incorporated into or fed in combination with chicken feed as a feed through formulation to control sand fly larvae. In some aspects, the insecticide comprises cyromazine and/or diflubenzuron. In some aspects, the insecticide comprises imidacloprid and/or fipronil In some aspects, the pesticide comprises cyromazine, diflubenzuron, imidacloprid, fipronil, and mixtures thereof.

Supplements useful in the present invention include a feed used with another feed to improve the nutritive balance or performance of the total. Supplements include compositions that are fed undiluted as a supplement to other feeds, offered free choice with other parts of an animal's ration that are separately available, or diluted and mixed with an animal's regular feed to produce a complete feed. Supplements include mineral blocks, salt licks, or feed additives, and can be in various forms including powders, tablets, boluses, liquids, syrups, pills, encapsulated compositions, and the like.

Illustratively, a mineral block lick delivery system can be used to deliver the insecticidal dose together with necessary vitamins and minerals used to maintain good live-stock health. Typically, the block is made up of urea 14% w/w, molasses 46% w/w, minerals 10% w/w, calcite powder 8% w/w, sodium bentonite 3% w/w, cottonseed meal 14% w/w, sodium chloride 5% w/w, and insecticide or IGR as desired, for example, 0.001% w/w to about 0.1% w/w.

Treats include compositions that are given to an animal to entice the animal to eat during a non-meal time, e.g., dog bones for canines. Treats may be nutritional the composition comprises one or more nutrients, and may have a composition as described above for food. Non-nutritional treats are also contemplated herein. The insecticide can be coated onto the treat, incorporated into the treat, or both.

The compositions provided herein can contain additional ingredients such as vitamins, minerals, fillers, palatability enhancers, binding agents, flavors, stabilizers, emulsifiers, sweeteners, colorants, buffers, salts, coatings, and the like known to skilled artisans. Stabilizers include substances that tend to increase the shelf life of the composition such as preservatives, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches.

Specific suitable amounts for each component in a composition will depend on a variety of factors such as the species of animal consuming the composition; the particular components included in the composition; the age, weight, general health, sex, and diet of the animal; the animal's consumption rate; level of insect infestation; and the like. Therefore, the component amounts may vary widely and may deviate from the proportions described herein.

The compositions comprise an amount of the one or more insecticides suitable for control of the bloodsucking parasite. The insecticide (or mix of insecticides) should be present at concentrations that are not toxic or otherwise deleterious to the health of the mammal being treated. In some embodiments, the insecticide is present in a range of about 0.05 mg/kg to about 5.0 mg/kg, for example, in an amount of about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.5 mg/kg, about 2.0 mg/kg, about 2.5 mg/kg, about 3.0 mg/kg, about 3.5 mg/kg, about 4 mg/kg, about 4.5 mg/kg, or about 5 mg/kg. In some embodiments, the formulation containing the one or more insecticides comprises about 0.001% to about 0.1% imidacloprid w/w, or about 0.01% to about 0.025% imidacloprid w/w, for example, about 0.001%, about 0.005% w/w, about 0.01% w/w, about 0.02% w/w, about 0.03% w/w, about 0.04% w/w, about 0.05% w/w, about 0.06% w/w, about 0.07% w/w, about 0.08% w/w, about 0.09% w/w, or about 0.1% w/w imidacloprid. In some embodiments, the formulation containing the one or more insecticides comprises about 0.005% to about 0.1% fipronil w/w, or about 0.01% to about 0.02% fipronil w/w, for example, about 0.005% w/w, about 0.01% w/w, about 0.02% w/w, about 0.03% w/w, about 0.04% w/w, about 0.05% w/w, about 0.06% w/w, about 0.07% w/w, about 0.08% w/w, about 0.09% w/w, or about 0.1% w/w fipronil.

In some embodiments, the insecticide is present in an amount such that a minimally effective concentration is present in the feces to control substantially all of the insect larvae. In some embodiments, the composition comprises the pesticide in an effective amount to control (kill) substantially all the adult blood sucking adult insects as well as substantially all the larvae present in the feces. “Substantially all” can include at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% of the insect larvae present in the feces and/or the adult blood sucking insect population. In some aspects, the insecticide is present in an amount of about 0.5 mg/kg to about 5 mg/kg.

At concentrations above 5 mg/kg, the treated animal should be given several days for withdrawal from the treatment before the milk produced by the animal is consumed by humans or before the animal is butchered for meat.

In a further aspect, provided herein is a kit comprising a composition suitable for oral administration animals for controlling ectoparasites. The kits comprise in separate containers in a single package or in separate containers in a virtual package, as appropriate for the kit component, an effective amount of the composition for controlling ectoparasites and instructions for how to combine the composition with a food product typically consumed by the animal. When the kit comprises a virtual package, the kit is limited to instructions in a virtual environment in combination with one or more physical kit components.

The examples below demonstrate the unusual rapid onset of insecticidal activity of fipronil and imidacloprid in oral formulations. Several trials were performed. One was a comparative feed through sand fly (P. argentipes) larval bioassay using rats (Rattus rattus) with fipronil and three other commonly used ectoparasitic control agents, eprinomectin, ivermectin, and diflubenzuron. Another trial was performed using sand rats (Psammomys obesus) as the carrier of sand flies (P. papatasi). Fecal samples were collected after three to seven consecutive days of administration of the trial products. The efficacy of the drugs administered during the blind study underwent testing in larval bioassays with both sand fly subspecies 1^{st} instar larvae.

In other embodiments, compositions and methods described herein further include ivermectin, abamectin, doramectin, emamectin, eprinomectin, or mixtures thereof. The ivermectin (or other like compound) controls the endoparasites, while the fipronil or imidacloprid kills both adult and larval bloodsucking ectoparasites. Fipronil and imidacloprid concentrations or percentages are as shown above; ivermectin percentages include about 0.001% w/w to about 0.1% w/w, though other ranges and concentrations are contemplated herein, for example about 0.001%, about 0.005% w/w, about 0.01% w/w, about 0.02% w/w, about 0.03% w/w, about 0.04% w/w, about 0.05% w/w, about 0.06% w/w, about 0.07% w/w, about 0.08% w/w, about 0.09% w/w, or about 0.1% w/w ivermectin. In some aspects, ivermectin is provided to an animal in about 0.01 mg/kg to about 1.0 mg/kg dose, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, or about 1.0 mg/kg dose. In some embodiments, the ivermectin is provided in an 80 mg dose per 400 kg body weight.

Surprisingly, the inventors have determined that administration of such rapid acting formulations not only control ectoparasites and endoparasites, but further, when administered to milk producing animals, causes the milk production to improve.

In some embodiments, the compositions described herein are administered to humans to control ectoparasites and/or endoparasites. In areas where the human population has little or no access to latrines, parasitic larvae thrive. It is contemplated herein that the inventive compositions and methods of using such compositions are useful in humans.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

The following examples are provided for illustrative purposes only and are not limiting to this disclosure in any way.

### Sand Flies

P. argentipes utilized in the studies were obtained from the Genesis Laboratories/Pestiscience facility located in Patna, India. The sand fly colony, which was founded from wild caught adult P. argentipes, was maintained in an insectary at 20-26° C. and approximately 80% humidity. Adult sand flies were regularly fed on immobilized rabbits and a 15% sugar solution was provided to maintain energy. Larval bioassays utilized one to two day old first instar larvae. Larvae were transferred to bioassay observation containers using a fine tip paintbrush. Sand flies used for adult bioassays were starved for 12 hours prior to exposure to treated B. bengalensis.

### Example 1

In the first study, a feed through study comparing diflubenzuron, fipronil, ivermectin, and eprinomectin was conducted on rats (Rattus rattus). Fecal samples were collected after three (3) consecutive days of administration of the trial products. The efficacy of the drugs administered during the blind study underwent testing in larval bioassays with P. argentipes 1^{st} instar larvae. Commercially available chicken feed was mixed with the appropriate concentrations of each insecticide compound and used as is in the trial.

Twelve (12) locally purchased rats (Rattus rattus) of mixed sex were utilized in a small study testing the efficacy of four feed through insecticides. Five treatment groups of two rats each were randomly identified and fed diets consisting of locally available chicken feed treated with one of the following compounds: diflubenzuron (0.048%), fipronil (0.015%), ivermectin (0.025%), and eprinomectin (0.01% and 0.025%). Two rats served as control and were fed only chicken feed. Rats were fed 20 g of their assigned diet daily at the same time, with daily consumption and spillage calculated for a period of three (3) days. Sand fly analyses were conducted simultaneously.

### Larval Bioassays

Feces were cleared the evening prior for collection on the mornings of day 0 post-feed through treatment to be utilized in larval bioassays. Collected feces were dried at approximately 40° C. in an oven then ground into a fine powder utilizing a pestle and mortar and frozen at −20° C. until larval bioassays were initiated.

Larval bioassay pots were prepared using 48 mm, 100 g round Dibbi jars. Dishes were prepared by burning several small holes into the bottom of the dish with a soldering iron and filling them with a small layer of plaster (¼ to ½ inch depth). Approximately 15-20 holes were punctured into the lids using 24 gauge needles. For simpler mortality counting, pots were quartered and each quadrant numbered using an ultra fine black Sharpie.

For each individual post-treatment bioassays, thirteen (13) dishes were loaded with 10 day old 1^{st} instar P. argentipes larvae; twelve (12) pots were provided with approximately 0.005 g of the treated feces, with two sample pots per treated bandicoot rat, and one overall control sample fed only standard larval diet. See Table 1 for sample/treatment correlations. Larvae were loaded into bioassay pots and provided with their designated treatment sample. Mortality counts of larvae were conducted every 24 hours post-treatment until 100% mortality, or pupation, was reached.

### Adult Bioassays

Adult sand fly bioassays were conducted on days 0, 5, 10 and 20 post-feed through treatment of Bandicota bengalensis. To conduct the bioassays, B. bengalensis were systematically anesthetized with 15 units Ketamine in an insulin syringe. The belly of each rat was shaved using an electric razor and a capsule containing 20 adult female and 5 adult male P. argentipes was affixed with medical tape. The capsules remained in place for one hour, covered with a light cloth to maintain warmth and reduce light. At the end of one hour, the capsules were removed. The intent was to transfer the capsules to the PestiScience laboratory where mortality observations would be conducted immediately, at 12-hour post feeding, and every 24-hours thereafter for up to 5 days post exposure, however zero blood feeding occurred, and thus no data was recorded.

TABLE 1
Sample ID and Treatment Correlation

| ID | Rat | Treatment |
|---|---|---|
| RR 1/2-1/2 | RR-01/02 | Control |
| RR 1/2-1/2 | RR-03/04 | Diflu. 0.048% |
| RR 1/2-1/2 | RR-05/06 | Fip. 0.015% |
| RR 1/2-1/2 | RR-07/08 | Eprino. 0.01% |
| RR 1/2-1/2 | RR-09/10 | Eprino. 0.025% |
| RR 1/2-1/2 | RR-11/12 | Ivermect. 0.025% |

Feeding of rats on treated food was good for all groups except those administered fipronil at concentrations of 0.025%. Over the initial 3 day treatment period, feeding declined dramatically (see Table 2). Thus, after day 3 consumption was measured, the treatment group was provided 2 extra days of treatment at a lower concentration of fipronil (0.015%); observed feeding over the two day period increased dramatically. Feeding the first day at the lower dosage was greater for both rodents than during the 3 previous treatment days, and almost equivalent to that of rodents on other treatments.

TABLE 2
Consumption of Insecticide Treated Feed by Rattus rattus

| | | | | | | Consumption | |
|---|---|---|---|---|---|---|---|
| | | | | | | after decrease | |
| | | | | | | in Fipronil | |
| | | | Consumption | | | concentration | |
| | | | Day | | Day | | Day |
| ID | Sex | Treatment | 1 | Day 2 | 3 | Day 4 | 5 |
| RR-1 | M | Control | 13 g | 14 g | 13 g | | |
| RR-2 | F | Control | 13 g | 13 g | 11 g | | |
| RR-3 | M | Diflu. 0.048% | 10 g | 17 g | 16 g | | |
| RR-4 | M | Diflu. 0.048% | 9 g | 15 g | 14 g | | |
| RR-5 | M | Fip. 0.015% | 9 g | 3 g | 2 g | 7 g | 7 g |
| RR-6 | M | Fip 0.015% | 5 g | 5 g | 1 g | 8 g | 9 g |
| RR-7 | M | Eprino. 0.01% | 10 g | 14 g | 13 g | | |
| RR-8 | M | Eprino. 0.01% | 13 g | 17 g | 12 g | | |
| RR-9 | F | Eprino 0.025% | 10 g | 7 g | 5 g | | |
| RR-10 | M | Eprino 0.025% | 12 g | 9 g | 3 g | | |
| RR-11 | M | Iverm. 0.025% | 9 g | 12 g | 7 g | | |
| RR-12 | M | Iverm. 0.025% | 9 g | 9 g | 6 g | | |

### Larval Bioassays:

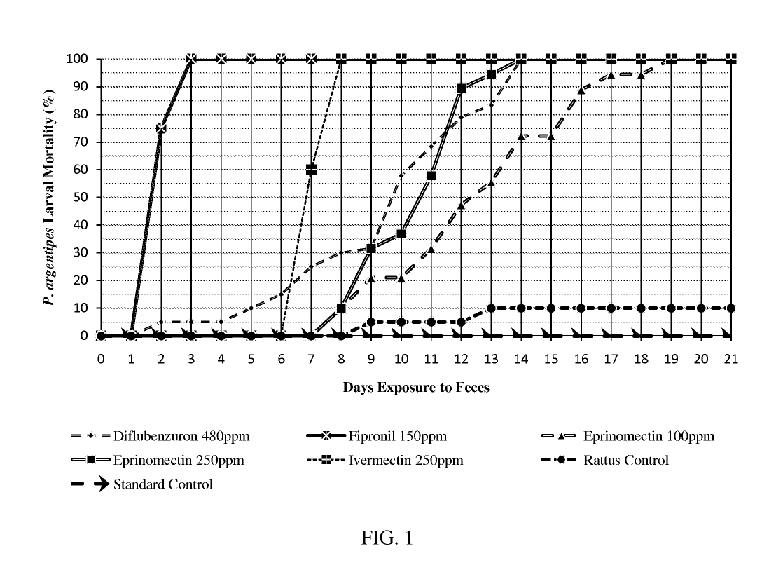

Total mortality for all treatment groups was exhibited within 20 days post treatment. Fipronil (0.015%) exhibited the fastest mortality, with all larvae dying by day 3 of observation. Ivermectin was the second most efficient with 100% mortality by day 8. Diflubenzuron and eprinomectin 0.025% exhibited complete mortality by day 14. And eprinomectin 0.01% exhibited full mortality by day 20. See FIG. 1.

Total mortality for all treatment groups was exhibited within 20 days post treatment. Fipronil (0.015%) exhibited the fastest mortality, with all larvae dying by day 3 of observation. Ivermectin (0.025%) was the second most efficient with 100% mortality by day 8. Diflubenzuron and eprinomectin 0.025% exhibited complete mortality only as late as day 14, while the commonly used lower dose of eprinomectin of 0.01% only exhibited full mortality at a much later day 20. (FIG. 1) Control (rodent): Zero (0%) mortality occurred during the first 8 days of observation. Day 9, one larvae (5%) died, and day 13 a second larvae died. Total control mortality on rodent feces was 10% (2 larvae).

TABLE 3
Mean (±SEM) insecticide consumption and survival
of P. argentipes larvae when exposed to feces of insecticide
treated R. rattus day-1 post treatment.

| | | | Mean ± SEM |
|---|---|---|---|
| | | P. argentipes | survival (days) |
| R. rattus | | larvae | of P. arg larvae |
| treatment groups | | mortality (%) | post-exposure |
| Diflubenzuron, | 19.44 mg ± 0.85 | 100 | 9.75 ± 0.71 |
| 480 ppm | | | |
| Eprinomectin, | 5.45 mg ± 0.25 | 100 | 12.65 ± 0.70 |
| 100 ppm | | | |
| Eprinomectin, | 3.95 mg ± 0.25 | 100 | 10.70 ± 0.38 |
| 250 ppm | | | |
| Fipronil, | 5.75 mg ± 0.80 | 100 | 2.25 ± 0.10 |
| 150 ppm | | | |
| Ivermectin, | 6.0 mg ± 0.00 | 100 | 7.40 ± 0.11 |
| 250 ppm | | | |

### Discussion

Although all insecticides demonstrated some level of efficacy against larval P. argentipes, fipronil resulted in the quickest mortality and longest lasting effectiveness of all of the compounds tested. Larvae exposed to feces of fipronil-treated animals demonstrated paralysis within 24 hours of exposure, likely due to the mode of action of the insecticide which blocks the GABAA-gated ion channels in the central nervous system (Ali et al., 1998; Gunasekara and Troung, 2007; NPIC, 2009). Quick knockdown of larvae was observed even when larvae were exposed to feces from rodents collected 20 days after the rodents were treated with fipronil. This level of efficacy was further exemplified by the efficacy of fipronil against bloodfeeding adult P. argentipes. When adult flies bloodfed on rodents that had been treated as much as 20 days previously, 100% mortality was observed at all treatment levels.

Palatability of fipronil was of concern during these trials. It was noted that the 250 ppm treatment level diet was not readily consumed by R. rattus. However, when the treatment level was reduced to 150 ppm, it was readily consumed. Additionally, B. bengalensis readily consumed fipronil treated diet at all treatment levels. Given these observations and the level of efficacy of the 100 ppm treatment level, palatability of a product utilizing this insecticide will likely be of no concern.

Based on the results of these trials, fipronil demonstrates the quickest knockdown and longest lasting efficacy of all of the insecticides examined.

### Example 2

A second trial was carried out using sand rats (Psammomys obesus) as the carrier of sand flies (P. papatasi) and imidacloprid as the insecticide. This rodent species was targeted since it serves as the source of blood meals for adult sand flies and its feces serves as a platform for larval development in Middle East ecosystems.

The test insecticide, imidacloprid at 250 ppm (0.025%) was incorporated into the feed together with orange, yellow, or green dye and fed to rats housed in standard laboratory caging. The treatment groups consisted of 6 males and 9 females; Controls: 3 males and 4 females with a body weight between 125-250 g.

DESIGN—Five sand rats were provided the reformulated diet for each of the treatment levels (50 ppm, 100 ppm, 250 ppm). Five sand rats were provided with control diets without imidacloprid. Two additional rats were provided with control diets containing green dye.

Samples for each imidacloprid-treated bait were evaluated by HPLC at Genesis Laboratories, Inc. for verification of imidacloprid levels. Samples were prepared by grinding in a UDY mill, followed by methanol extraction. The supernatants were decanted and the extraction procedure was repeated two additional times with fresh aliquots of methanol. An aliquot of each sample was filtered through a 0.20 μm syringe filter into an HPLC vial for analysis in comparison to prepared standards.

Feces were collected from the Alpha-Dri bedding for the last 5 days of the 7 day treatment period. Feces were weighed and transferred to a plastic bag labeled with the animal's cage identification number and stored frozen at −20° C.

Bioassays of the larval groups comprised: four imidacloprid treatments (0, 50, 100, 250 ppm) (number of replicates per assay n=5), one control group (n=8), and one green dye treatment (n=2). These bioassays were conducted with 1^{st} and 2-3^{rd} instar larvae and were repeated three times. Bioassays were conducted in 6 well culture plates (Corning, Inc) with 5 ml of plaster of Paris in the bottom of each well. The plaster was saturated with distilled water before the experiment, and was blotted with filter paper to remove standing water immediately before use. The effect of imidacloprid treatment on 1^{st} and 2^{nd}-3^{rd} instar larvae was tested. The control group was provided using standard larval diet. This allowed comparison of sand fly survival between those feeding on feces from sand rats that fed on non treated feed and those fed on standard sand fly diet. First instar larvae were obtained by adding 50 eggs to each well and allowed to hatch and fed on regular diet for 2-3 days. At this time ca. 0.1 g of crushed pellets or control feed was added. Second-3^{rd} instar larvae were obtained by letting 1^{st} instar larvae grow on standard diet in the wells until moulted. At this, time crushed pellets or control feed (0.1 g) was added. Due to variation in hatching and growth time this resulted in a mix of 2^{nd} and 3^{rd} instar larvae at approximately 1:1 ratio. The wells of the plate were covered with parafilm which was punctured with a needle to allow for ventilation. The wells were kept in a humidified room (26° C./75% RH) inside a covered tube which contained a saturated sponge. The container was placed in an environmental chamber at 28 C, 90% RH, and a photoperiod of 14:10 (L:D) h. Larval mortality was recorded daily; larvae were considered dead if they did not respond within 15 s to prodding with a blunt probe. Alimentation was noted by observation of the presence of frass in the vials and dark material in the guts of the larvae. All larvae were observed for abnormal behavioral and morphological characteristics.

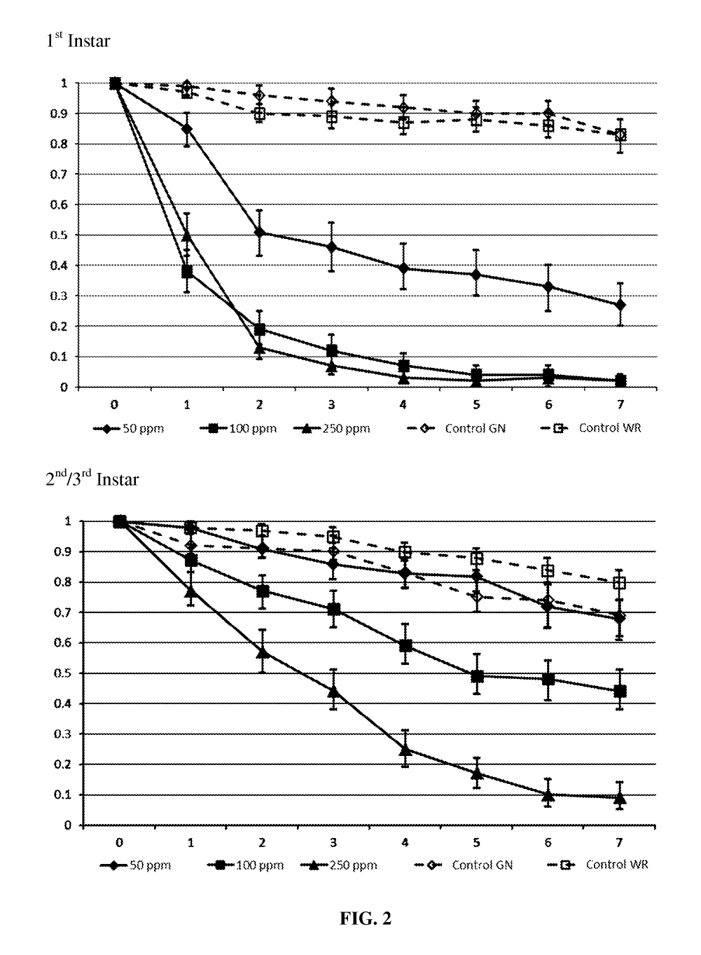

All sand rats accepted the diets containing imidacloprid without any apparent health abnormalities. Sixteen sand rats gained weight during the trials. No sand rats lost more than 5 g (3% body weight), which was well within typical weekly weight variations. Survivorship for 1^{st} instar larvae cultured on feces from each treatment group ranged from 90% for controls, to <5% by day 5 for larvae on feces from sand rats administered 100 ppm and 250 ppm imidacloprid (FIG. 2). Survivorship for 2^{nd}/3^{rd} instar larvae cultured on feces from each treatment group ranged from 80% for controls, to 10% by day 7 for larvae on feces from sand rats administered 250 ppm imidacloprid (See FIG. 2). This study demonstrated that sand rats will eat baits containing imidacloprid without apparent health abnormalities, and most sand rats gained weight on this diet. There was no significant difference (P=0.1199) in the fecal production, and presumably, food consumption, between treatment groups. The key information from the sand fly larvae bioassay is that sand rats fed diets of 100 ppm and 250 ppm produced feces that were rapidly and highly larvicidal for 1^{st} instar larvae. Diets containing 250 ppm imidacloprid resulted in feces for which there was 90% mortality by seven days. See FIG. 2.

### Example 3

Nine wild B. bengalensis were captured using baited Tomahawk (Tomahawk Live Trap Co, Tomahawk, Wis.) and Sherman live animal traps (H.B. Sherman Traps, Tallahassee, Fla.). B. bengalensis was chosen for testing as it is one of the foremost agricultural pests in Bihar, living in close proximity to both human households and livestock. The seasonal extremes within their elaborate burrow systems are less drastic than outside and the average monthly relative humidity exceeds 89%, thereby providing a potentially ideal microclimate for sand fly oviposition and larval development (Mitchell, 1971).

No discrimination or preference for sex was emphasized, however juvenile, small, and apparently unhealthy animals were not included in the studies. All animals were treated with 2 drops of 8.8% imidacloprid and 44% permethrin topical treatment (K9 Advantix®, Bayer, Shawnee Mission, Kans.) to clear animals of potential ectoparasites such as fleas, ticks, and lice. Prior studies indicate zero residue of imidacloprid in blood three days post oral treatment and permethrin, which is not readily absorbed by the skin, is readily metabolized and the majority of the product excreted by rodents within 48 hours of oral treatment (FAO and WHO, 1999; unpub. data). Previous data by Borchert and Poche (2003) demonstrated no residues in the blood stream of rodents after three days. Based upon these factors, feed-through studies were initiated no sooner than three days post application of topical treatment. All test animals were housed individually in wire mesh cages with ceramic food dishes and individual water bottles.

The B. bengalensis fipronil study was conducted in two portions. In the first of the two segments, three randomly selected B. bengalensis were offered a fipronil (250 ppm) treated diet at 25 g daily. One rat served as control and was fed an untreated diet. In the second portion of the study, the remaining animals were randomly divided into two groups of two. Two individuals in the first group were offered 25 g of 100 ppm fipronil-treated feed each and two rats in the second group were each offered 25 g of 50 ppm fipronil-treated diet. One rat served as control and was fed an untreated diet.

Test animals were provided treated feed for two consecutive days. Diets were prepared by utilizing locally available poultry feed treated with technical grade fipronil to predetermined concentrations. Consumption was calculated daily, feed refilled to a predetermined level of 25 g, and feces cleared. At the end of the second day, treated feed was cleared, final consumption weighed, and feed replaced with untreated locally available poultry feed. Feces were collected, noted as day-1 post-treatment and properly stored for use in assays. Observation of test animals continued for twenty days post-treatment, with additional collection of feces for testing conducted 5, 10, and 20 days post-treatment. On days when fecal collection occurred, all newspaper/bedding was replaced at 0800 hours and feces collected at the end of the day for preservation. Collected feces were dried overnight at approximately 40° C., ground into a fine powder with a pestle and mortar and frozen at −20° C. until larval bioassays were initiated.

### Larval Bioassays

Larval bioassay jars were prepared using 48 mm, 100 g round Dibbi jars (Pearlpet, Pearl Polymers LTD., New Delhi, India). Jars were prepared by burning three small holes into the bottom of the container with a soldering iron. A thin layer (approximately 5 mm) of plaster of Paris was cast on the bottom and wetted to ensure humidity and softening of the test diet. To simplify mortality counts, the plaster was quartered, and each quadrant numbered one through four, using an ultra fine black marker. The lids of the jars were punctured with 15-20 small holes using a heated 24-gauge hypodermic needle.

Each bioassay jar was loaded with ten one to two day old first instar P. argentipes larvae. Approximately 5 mg treated feces were sprinkled evenly over the plaster. Larval bioassay samples were maintained in a controlled environment at approximately 24° C. with relative humidity maintained at approximately 80% through daily moistening of paper towels underneath the jars. Any observed mold or mites were removed during each day's observation. Each bioassay group included one control jar with larvae provided standard larval food consisting of rabbit feed, rabbit pellets, and dried chicken blood which were mixed, dried, composted, and crushed. Mortality counts of larvae were conducted every 24 hours post-exposure until 100% mortality or pupation was observed. Larvae were considered dead if no physical response was observed within 15 seconds of light stimulation with a blunt probe.

### Adult Bioassays.

Adult sand fly bioassays were conducted 1, 5, 10 and 20 days post feed-through treatment of B. bengalensis. B. bengalensis were anesthetized with 15 units of ketamine (KetaJet 50, SterFil Laboratories Pvt. Ltd., Ankleshwar, India) via intramuscular injection. The belly of each rat was shaved using an electric razor and a mesh covered plastic capsule (20 mm diameter, 25 mm height) containing 20 adult female and 5 adult male P. argentipes was affixed to the shaved area with medical tape. The capsules had about 10 small holes burned into the top with a heated 24 gauge needle. Capsules remained in place for one hour, covered with a light cloth to maintain warmth and reduce light. At the end of one hour, the capsules were removed and observations for mortality conducted immediately, at 12 hours post-feeding, and every 24 hours thereafter for up to 5 days post-exposure. Partially fed sand flies were included in analyses as “bloodfed” specimens. Unfed sand flies were removed, and bloodfed sand flies were observed collectively by treatment group.

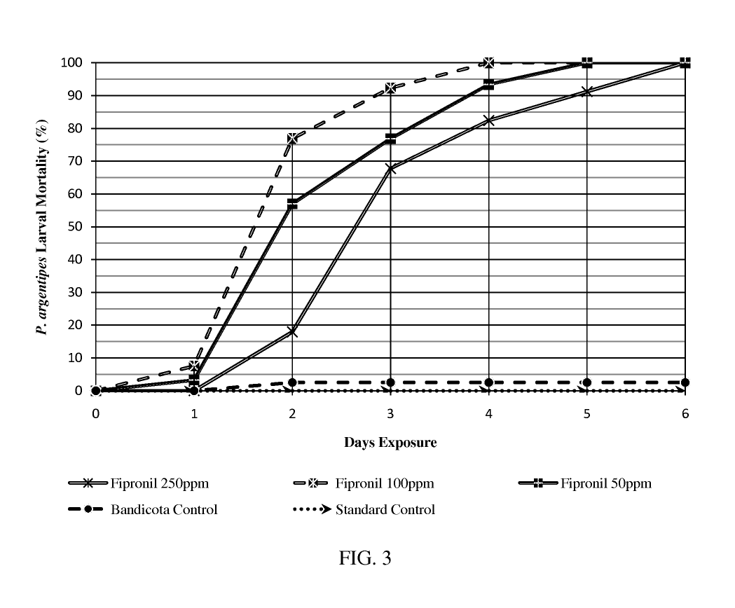

Results of larval bioassays conducted in Trial 2 are summarized as follows. When exposed to feces of fipronil-treated B. bengalensis collected on the day following treatment, larval P. argentipes mortality was observed at low levels after 1 day of exposure for the 50 ppm (4% mortality) and 100 ppm (8% mortality) treatment levels. Mortality was observed after 2 days of exposure for the 250 ppm treatment level (18% mortality). 100% mortality was achieved after 4 days of exposure for the 100 ppm treatment level, after 5 days of exposure for the 50 ppm treatment level, and after 6 days of exposure for the 250 ppm treatment level. These results are shown in FIG. 3.

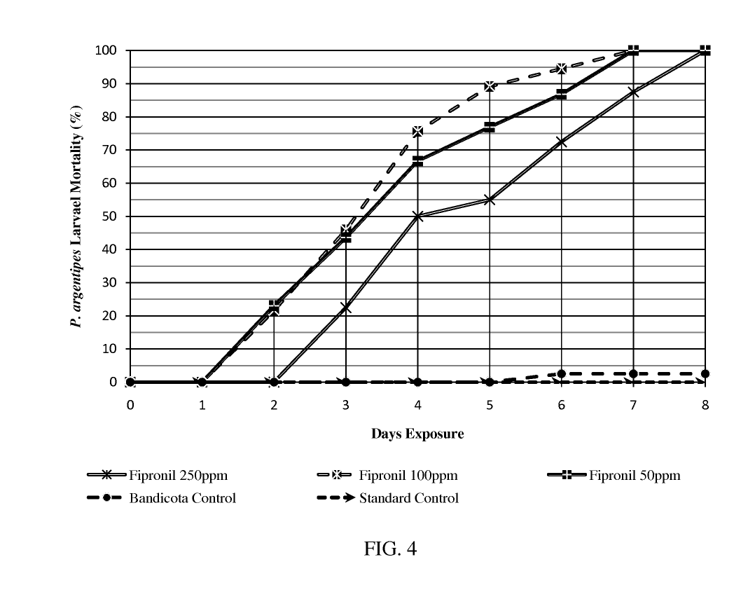

FIG. 4 shows mortality of P. argentipes larvae exposed to feces of fipronil-treated B. bengalensis collected 5 days after treatment. Similar results were demonstrated, with mortality first observed for the 50 ppm (23% mortality) and 100 ppm (22% mortality) treatment levels after 2 days of exposure to the treated feces, and after 3 days of exposure for the 250 ppm treatment level (23% mortality). 100% mortality was observed for both the 50 ppm and 100 ppm treatment levels after 7 days of exposure, and after 8 days of exposure for the 250 ppm treatment level.

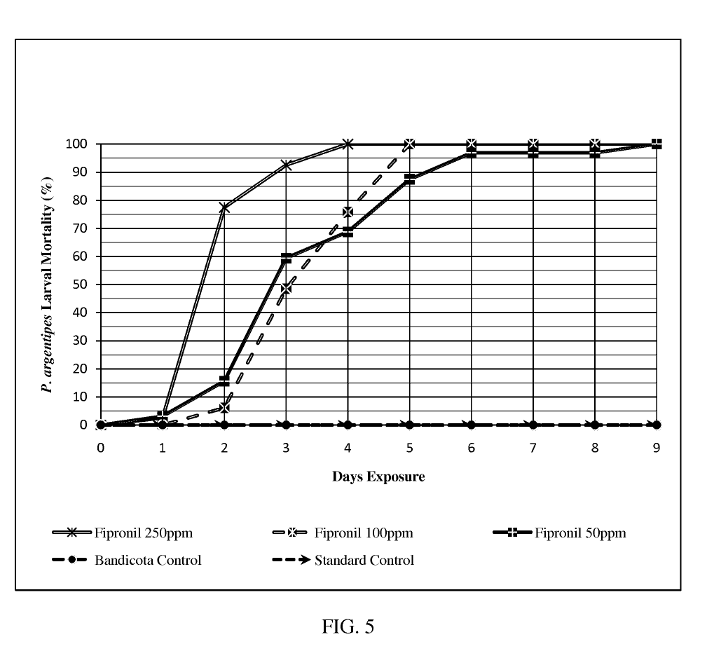

When P. argentipes larvae were exposed to feces of fipronil-treated B. bengalensis collected 10 days after treatment, 78% mortality was observed for the 250 ppm treatment group after only 2 days of exposure. A lower level of mortality was observed for the 50 ppm (16% mortality) and 100 ppm (6% mortality) treatment groups after 2 days of exposure. 100% mortality was achieved for the 250 ppm treatment group after 4 days of exposure, after 5 days of exposure for the 100 ppm treatment group, and after 9 days of exposure for the 50 ppm treatment group. It should be noted, however, that 95% mortality was achieved for the 50 ppm treatment group after 6 days of exposure. These results are shown in FIG. 5.

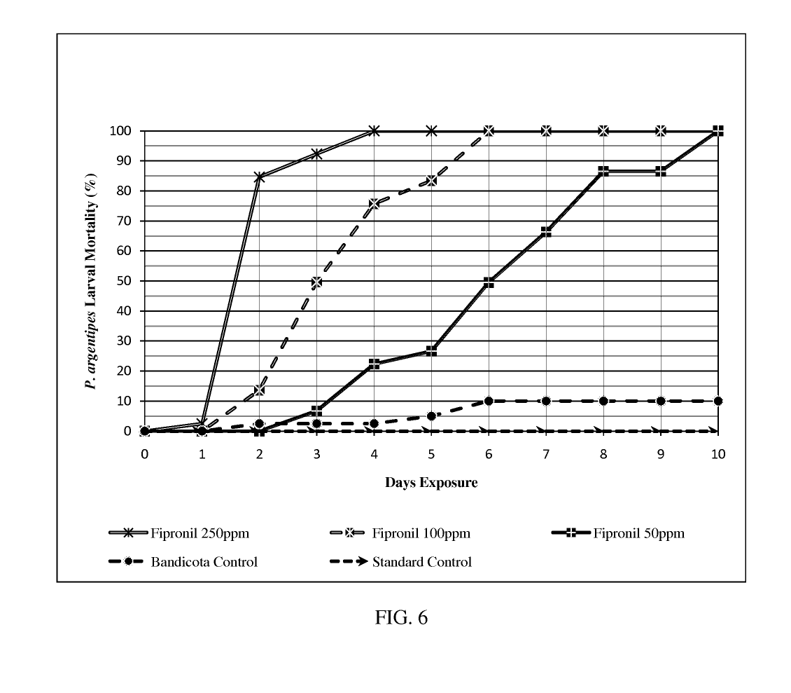

FIG. 6 shows mortality of P. argentipes larvae exposed to feces of fipronil-treated B. bengalensis collected 20 days after treatment. After 2 days of exposure, 85% mortality was observed in the 250 ppm treatment group. Lower mortality levels were demonstrated after 2 days of exposure for the 100 ppm treatment group (14%) and after 3 days of exposure for the 100 ppm treatment group (7%). 100% mortality was achieved for the 250 ppm treatment group after 4 days of exposure, after 6 days of exposure for the 100 ppm treatment group, and after 10 days for the 50 ppm treatment group.

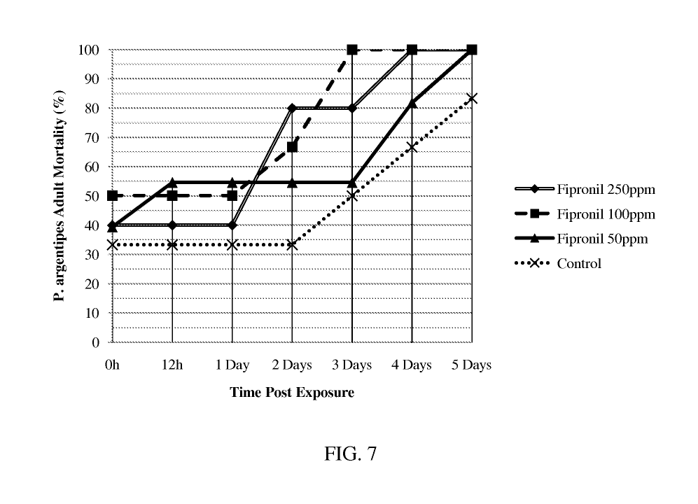

Adult bioassays demonstrated 100% mortality across treatment groups when sand flies were allowed to bloodfeed on rodents 1 and 5 days after the rodents were treated. Sand flies exposed to B. bengalensis from all treatment groups (50 ppm, 100 ppm, and 250 ppm) on day 1, and sand flies exposed to rats from the 250 and 100 ppm treatment groups on day 5 were dead by the end of the one hour exposure period. Sand flies exposed to rodents from the 50 ppm treatment group on day 5 required 24 hours to exhibit 100% mortality. When adult P. argentipes were allowed to bloodfeed on B. bengalensis 20 days after the rodents were treated, 100% mortality was observed at the 100 ppm level 3 days after the flies were exposed for 1 hour. For the 250 ppm treatment level, 100% mortality was observed after 4 days, and for the 50 ppm treatment level, 100% mortality was observed 5 days after exposure to treated animals. FIG. 7 shows the results of the day 20 adult bioassays.

TABLE 4
Mean (±SEM) survival of P. argentipes larvae
when exposed to feces of fipronil treated B. bengalensis
collected 1, 5, 10 and 20 days post-treatment.

| | Mean ± SEM survival (days) of P. argentipes | | | |
|---|---|---|---|---|
| Fipronil | larvae during exposure to treated feces | | | |
| Dosage | D-1 | D-5 | D-10 | D-20 |
| 250 ppm | 3.37 ± 0.20 | 5.13 ± 0.27 | 2.28 ± 0.10 | 2.20 ± 0.10 |
| 100 ppm | 2.23 ± 0.11 | 3.73 ± 0.23 | 3.70 ± 0.16 | 3.82 ± 0.21 |
| 50 ppm | 2.66 ± 0.18 | 4.03 ± 0.27 | 4.06 ± 0.29 | 6.36 ± 0.36 |

### Example 4

The test substance was given to the cattle orally as a single application. The oral treatment was by hand. Because the oral treatment was delivered in capsule form the dosage was very precise.

The animals were observed for a minimum period of 8 weeks based on the residual activity of the product following the dose application. The observation period can be extended, if necessary based on drug persistence data and residue analysis. The first day of dose application was designated as day 0.

The four dose levels of fipronil for the treatment groups used in this study were as follows: 0.5, 1.0, 2.0 and 4.0 mg/kg. These were weighed with an analytical balance to the nearest 0.01 g and presented in gelatin capsules.

Body weights were recorded individually for all animals during randomization, at the beginning of each week (Monday) during the study period, and at the termination of the study.

Collection of feces was performed at pre-dosing (day 0) and days 1, 3, 5, 7, 14, 21, and 28 following administration. Feces were sampled using arm length veterinarian sterile glove and about 50 g of feces was placed into individually label plastic jars. Samples were stored in a −20° C. freezer.

### Adult Sand Fly Bioassay

A breeding colony of P. argentipes was established during 2009 and is situated in Patna. The colony contains an average of 10,000 sand flies. Adults and larvae are used routinely for studies and are maintained under controlled temperature and humidity conditions.

Adult sand flies used in this study were transferred to a mud plastered but built next to the cattle shed. This was to acclimate lab-ready sand flies to natural environmental conditions in which the study was to be conducted. The sand flies were kept in 0.4 m³ cloth mesh enclosures.

Adult sand fly bioassays were conducted on days 1, 3, 5, 7, 14, 21, and 28 after oral administration of fipronil. Sand flies used were between 3 and 6 days post-emergence at the time of the assay and were fasted for 12 hours before each test. Sand flies were counted and placed in a sand fly feeding capsule (10 cm diameter×2 cm deep). The top of the capsule had a minimum of 15, 0.5 mm holes burned through the container to facilitate air flow. The bottom of the capsule had a cloth mesh (<1 mm) so that sand flies could feed through the cloth to obtain a blood meal. Sand flies were transferred into the capsules using a suction pipette and an insertion slot made into the side of the capsule. In each capsule, 20 female and 5 male sand flies were placed.

Two sand fly feeding capsules were used per cow. Each capsule was held in place using elastic bandages and was placed onto an area shaved on the belly of the cow so that the skin was fully exposed and to enable sand fly feeding. Sand flies were allowed to feed for 60 minutes and were monitored closely throughout the feeding period.

Immediately following feeding, the flies were examined for mortality then transferred into a small cage and examined for post-feeding mortality. Separate cages were kept for flies fed each day on each cow. Mortality was examined after 12 hours, then every 24 hours each day.

After 6 days the flies were grouped according to the treatment level on which they were fed and transferred into a larger cage containing other flies fed on the same treatment group. These flies were blood fed on a rabbit as specified in an SOP.

### Larval Bioassays

Larval bioassay jars were prepared using 48 mm, 100 g round Dibbi jars (Pearlpet, Pearl Polymers LTD., New Delhi, India). Jars were prepared by burning three small holes into the bottom of the container with a soldering iron. A thin layer (approximately 5 mm) of plaster of Paris was cast on the bottom and wetted to ensure humidity and softening of the test diet. To simplify mortality counts, the plaster was quartered, and each quadrant numbered one through four, using an ultra fine black marker. The lids of the jars were punctured with 15-20 small holes using a heated 24-gauge hypodermic needle.

Each bioassay jar was loaded with ten one to two day old first instar P. argentipes larvae. Approximately 5 mg treated feces were sprinkled evenly over the plaster. Larval bioassay samples were maintained in a controlled environment at approximately 24° C. with relative humidity maintained at approximately 80% through daily moistening of paper towels underneath the jars. Any observed mold or mites were removed during each day's observation. Each bioassay group included one control jar with larvae provided standard larval food consisting of rabbit feed, rabbit pellets, and dried chicken blood which were mixed, dried, composted, and crushed. Mortality counts of larvae were conducted every 24 hours post-exposure until 100% mortality or pupation was observed. Larvae were considered dead if no physical response was observed within 15 seconds of light stimulation with a blunt probe.

### Larval Sand Fly Bioassay

At pre dosing (day 0), 1, 3, 5, 7, 14, 21, and every 7 days after as needed feces samples were collected from the study animals for residue analysis by HPLC with fluorescent detector, or other appropriate analytical equipment.

### Results

### Cattle Dosing

There were no observed adverse effect from dosing the cattle at the four levels of fipronil, 0.5, 1.0, 2.0, and 4.0 mg/kg body weight. Use of the elastic band to hold the sand fly capsules onto the shaved areas of the cow belly proved to be effective and did not appear to alter the behavior of the cattle. None of the treated and control cattle were disrupted by the one hour exposure of sand flies to the animals.

### Adult Sand Fly Bioassay

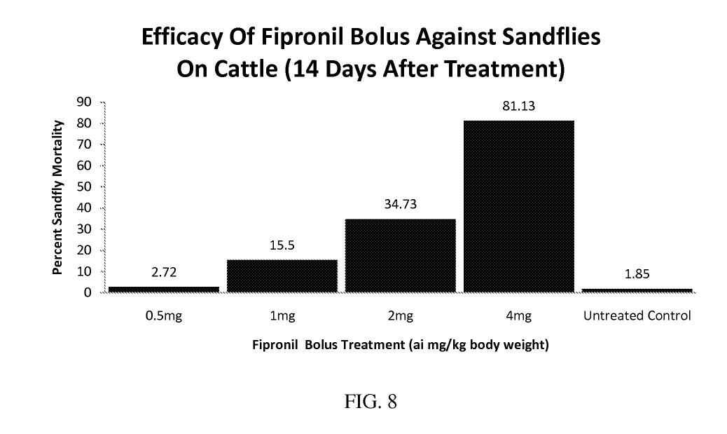

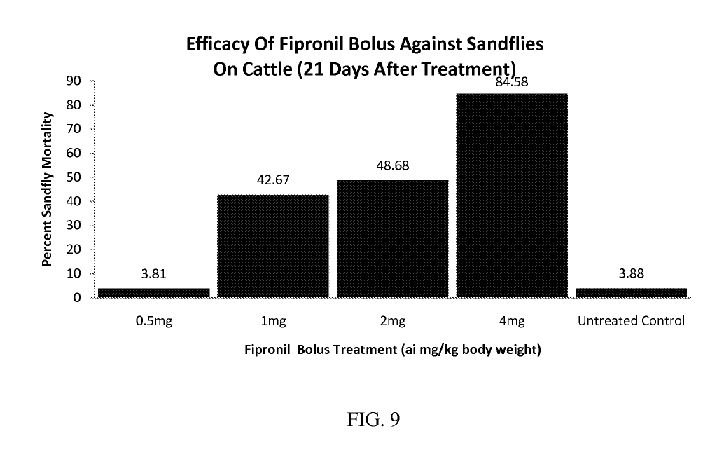

The capsules used to contain sand flies in this study worked well. They were easy to handle and the elastic bandages held them closely to the animal skin. Adult sand fly mortality data from days 14 and 21 are presented in FIGS. 8 and 9. As would be expected, mortality increased with dose level. These data reflect that use of fipronil as a systemic to control adult sand flies has merit, although high levels of control were not attainable without having an impact on milk residues.

### Larval Bioassay

Table 5 presents the data for days 1, 3, 5, 14, and 21 after administration of the fipronil bolus. For day 3 post-treatment 100% sand fly mortality was attained by day 4. As the fipronil was slowly excreted and metabolized it require more time to eliminate the larvae. By Day-21 it required feeding on the treated feces for longer periods as shown with the 2 and 4 mg/kg response of 12 and 10 days respectively.

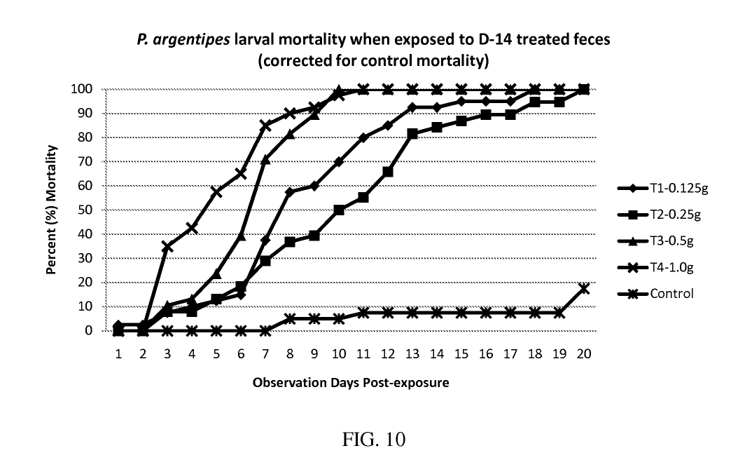

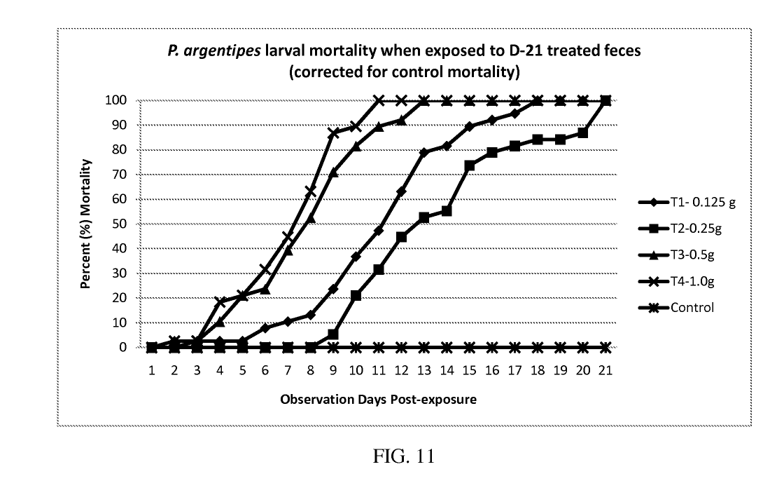

Detailed responses to sand fly larval mortality over time and dose levels are presented in FIGS. 10 and 11. At all dose levels, 100% larval mortality was obtained over the 21-day study, indicating that fipronil is an excellent drug for control of sand fly larvae.

TABLE 5
Mortality in larval P. argentipes sand flies fed treated cattle
feces after a single oral dose of fipronil.
Days after dosing to attain 100% mortality in sand flies

| Dose mg/kg | 1 | 3 | 5 | 14 | 21 |
|---|---|---|---|---|---|
| 0.5 | 5.5 | 6.0 | 9.5 | 17 | 15.5 |
| 1.0 | 4.5 | 4.0 | 9.0 | 20 | 16.5 |
| 2.0 | 4.0 | 4.0 | 7.0 | 10 | 12.0 |
| 4.0 | 4.0 | 4.0 | 6.5 | 11 | 10.0 |

When introducing elements or features of embodiments herein, the articles “a”, “an”, “the” and “said” are intended to mean that there are one or more of such elements or features. The terms “comprising”, “including”, and “having” are intended to be inclusive and mean that there may be additional elements or features other than those specifically noted. The phrase “consisting essentially of” refers to the specified materials or steps “and those that do not materially affect the basic and novel characteristic(s)” of the claimed subject matter. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

The description of the disclosure is merely exemplary in nature and, thus, variations that do not depart from the gist of the disclosure are intended to be within the scope of the disclosure. Such variations are not to be regarded as a departure from the spirit and scope of the disclosure.

Certain embodiments of the disclosure are as follows:

1. A composition comprising fipronil or imidacloprid for oral administration to animals for controlling ectoparasites.

2. The composition of claim 1, wherein the formulation comprises imidacloprid at a concentration between about 0.001% and about 0.1%.

3. The composition of claim 1, wherein the formulation comprises imidacloprid at a concentration between about 0.01% and about 0.025%.

4. The composition of claim 1, wherein the formulation comprises fipronil between about 0.005% and about 0.1% w/w.

5. The composition of claim 1, wherein the formulation comprises fipronil between about 0.01% and about 0.02% w/w.

6. A method of controlling ectoparasites comprising administration of an oral composition comprising fipronil or imidacloprid to a mammal.

7. The method of claim 6, wherein the composition is administered by incorporating the formulation into the mammal's feed.

8. The method of claim 6, wherein the composition is administered as a bolus.

9. The method of claim 6, wherein the composition is administered in a salt lick.

10. The method of claim 6, wherein the composition is administered in a mineral supplement.

11. The method of claim 6, wherein the composition is administered by incorporating the formulation into a supplement.

12. The method of claim 6, wherein the composition comprises imidacloprid between about 0.001% and about 0.1%.

13. The composition of claim 1, wherein the composition comprises imidacloprid between about 0.01% and about 0.025%.

14. The method of claim 6, wherein the composition comprises fipronil between about 0.005% and 0.1% w/w.

15. The method of claim 6, wherein the composition comprises fipronil between about 0.01% and 0.02% w/w.

16. The method of claim 6, wherein the mammal is avian, ovine, hircine, caprine, or bovine.

17. The method of claim 6, wherein the mammal is murine.

18. The method of claim 6, wherein the mammal is feline or canine.

19. The method of claim 6, wherein the ectoparasite is a sand fly.

20. The method of claim 6, wherein the ectoparasite is a mosquito.

21. The method of claim 6, wherein the ectoparasite is a tsetse fly.

22. The method of claim 6, wherein the ectoparasite is a tick.

23. The composition of claim 1, further comprising ivermectin, abamectin, doramectin, emamectin, eprinomectin, or mixtures thereof.

24. The method of claim 6, wherein the formulation further comprises ivermectin, abamectin, doramectin, emamectin, eprinomectin, or mixtures thereof.

25. A method of controlling sand fly larvae, the method comprising administering an insecticidal composition to a chicken, wherein the insecticidal composition comprises an insecticide selected from the group consisting of fipronil, imidacloprid, cyromazine, diflubenzuron, and mixtures thereof.

26. A composition comprising fipronil and a compound selected from the group consisting of ivermectin, abamectin, doramectin, emamectin, and eprinomectin for oral administration to animals for controlling ectoparasites, endoparasites, and parasitic larvae.

What is claimed is:

1. A composition for controlling ectoparasites, endoparasites, and larvae thereof, in milk-producing mammals, wherein said composition comprises fipronil or imidacloprid, and at least one compound selected from the group consisting of: ivermectin, abamectin, doramectin, emamectin, and eprinomectin; wherein said milk-producing mammals exhibit improved milk production.

2. The composition of claim 1, further comprising imidacloprid in an amount between 0.001% and 0.1% w/w.

3. The composition of claim 1, further comprising imidacloprid in an amount between 0.01% and 0.025% w/w.

4. The composition of claim 1, further comprising fipronil in an amount between 0.005% and 0.1% w/w.

5. The composition of claim 1, further comprising fipronil in an amount between 0.01% and 0.02% w/w.

6. A method of controlling ectoparasites, endoparasites, and larvae thereof, in milk-producing mammals comprising administration of an oral composition according to claim 1.

7. The method of claim 6, wherein the composition is administered by incorporating the formulation into the mammal's feed or a supplement selected from the group consisting of a mineral block, treat, and salt lick.

8. The method of claim 6, wherein the composition is administered as a bolus.

9-11. (canceled)

12. The method of claim 6, further comprising imidacloprid in an amount between 0.001% and 0.1% w/w.

13. The method of claim 6, further comprising imidacloprid in an amount between 0.01% and 0.025% w/w.

14. The method of claim 6, further comprising fipronil in an amount between 0.005% and 0.1% w/w.

15. The method of claim 6, further comprising fipronil in an amount between 0.01% and 0.02% w/w.

16. The method of claim 6, wherein the mammal is selected from the group consisting of: ovine, hircine, caprine, bovine, simian, canine, equine, asinine, feline, murine, cameline, camelid, leporine, macropodine, human, and porcine.

17-18. (canceled)

19. The method of claim 6, wherein the ectoparasite is selected from the group consisting of: fleas, lice, ticks, sand flies, deer flies, horse flies, stable flies, mosquitoes, bedbugs, blowflies, louseflies, blackflies, tsetse flies, bloodsucking conenose, and mites.

20-26. (canceled)

27. The composition of claim 1, further comprising ivermectin.

28. The composition of claim 27, further comprising ivermectin in an amount between 0.001% and about 0.1% w/w.

29. The composition of claim 1, comprising fipronil and ivermetcin.

30. The composition of claim 1, further comprising at least one additional ingredient selected from the group consisting of: vitamins, minerals, fillers, palatability enhancers, binding agents, flavors, stabilizers, emulsifiers, sweeteners, colorants, buffers, salts, and coatings.

31. The method of claim 6, further comprising ivermectin.

32. The method of claim 31, further comprising ivermectin in an amount between 0.001% and 0.1% w/w.

33. A kit comprising a composition for controlling ectoparasites, endoparasites, and larvae thereof, in milk-producing mammals, using the composition according to claim 1 and instructions for administration thereof.
